(19) United States
(12) Patent Application Publication
(10) Pub. No.: US 2026/0033718 A1
(43) Pub. Date: Feb. 5, 2026

(54) Eye Imaging Device

(71) Applicants: Paul J. Lee, Clarence, NY (US); Ella Clare Lee, Clarence, NY (US); Kalyan Krishna Ratnakaram, Williamsville, NY (US); Alyssa Rose Stathopoulos, East Amherst, NY (US)

(72) Inventors: Paul J. Lee, Clarence, NY (US); Ella Clare Lee, Clarence, NY (US); Kalyan Krishna Ratnakaram, Williamsville, NY (US); Alyssa Rose Stathopoulos, East Amherst, NY (US)

(21) Appl. No.: 18/386,175

(22) Filed: Nov. 1, 2023

Related U.S. Application Data
(60) Provisional application No. 63/421,198, filed on Nov. 1, 2022.

(51) Int. Cl. A61B 3/12; A61B 3/15

(52) U.S. Cl. CPC A61B 3/12; A61B 3/156

(57) ABSTRACT

A chamber houses a camera, light sources, and an onboard computer. The camera may be an infrared camera. Infrared light source fibers feed into a circumferential adapter ring to illuminate the inside of the eye (retina). The adapter ring contains a central opening through which it fits around the camera lens. The adapter ring contains openings along the circumference for infrared fibers to provide a light source. Another light source is provided for illumination of the external surface of the eye adjacent to the camera.

### CROSS-REFERENCE TO RELATED APPLICATION

The present application claims priority benefit from U.S. Provisional Patent Application No. 63/421,198 filed on Nov. 1, 2022, and entitled “Eye Imaging Device,” which is incorporated herein by reference.

The present invention provides a device and method for taking external and dilated internal (retinal) photographs of the eye without pharmacologic dilation.

### SUMMARY OF THE INVENTION

A darkened chamber houses a camera, light sources and an onboard computer. The camera may be an infrared camera. Infrared light source fibers feed into a circumferential adapter ring to illuminate the inside of the eye (retina). The adapter ring contains a central opening through which it fits around the camera lens. The adapter ring contains openings along the circumference for infrared fibers to provide a light source. Another light source is provided for illumination of the external surface of the eye adjacent to the camera.

A darkened chamber around the eye can take the shape of a virtual reality headset that covers both eyes. Any device that covers the eye(s) while allowing enough room for the camera and miniaturized onboard computer to fit into the enclosure may be suitable. The device may only cover one eye as the other eye can be closed. Miniature camera(s) such as an infrared camera pointing towards the subject's eye(s) may be used. The camera component is contained within the chamber. An adapter ring fits around the camera lens. The adapter ring has holes in which infrared light fibers are attached. Various adapter designs which have holes drilled at converging and diverging angles may be used. This feature allows for diffusion of the focal illumination.

Fiber optic cables of any light source type, such as but not limited to infrared light, may be fed into the adapter ring to provide the light source.

Additional light sources to illuminate the external eye surface may be provided to take images of the external surface of the eye. The images may be sent to an onboard miniaturized computer such as raspberry pi that may be connected to the camera. The device may connect via bluetooth to an external internet server. Additionally, images can be sent to external systems with our without wires for processing. For example, bluetooth-enabled cameras can be used to send images to an external processing system which may not be the on-board device.

Dilation may be achieved by placing the eye in a darkened chamber, such as virtual reality goggles, or in smaller cases surrounding the eye, such as larger swim goggles to accommodate the components. Infrared light fibers and infrared cameras are used to compensate for the lack of visible light. A secondary light source also exists to provide illumination on the external surface of the eye of the user. The images may be uploaded to an external server via a connection to the internet automatically and diagnosis is obtained via human or automated algorithms.

### BRIEF DESCRIPTION OF THE FIGURES

FIG. 1 is a perspective view of one embodiment of the device of the present invention.

FIG. 2 is a top plan view of the device shown in FIG. 1.

FIG. 3 is a front elevation view of the device of the present invention.

FIG. 4 is a front elevation view of the device with the camera removed for clarity.

FIG. 5 is a front perspective view of the device with the front cover removed.

FIG. 6 is a front elevation view of the device with the lenses removed for clarity.

FIG. 7 is a front elevation view of the device with the lenses removed.

FIG. 8 is a detailed view of a portion of FIG. 7.

FIG. 9 is a perspective view of an assembly of the present invention.

FIG. 10 is a another perspective view of the assembly shown in FIG. 9.

FIG. 11 is a perspective view of the adapter ring assembly of the present invention.

FIG. 12 is a front elevation view of the adapter ring.

FIG. 13 is a side elevation view of the camera assembly of the present invention.

FIG. 14 is a front elevation view of the assembly of FIG. 13.

### DESCRIPTION OF THE PREFERRED EMBODIMENTS

At the outset, it should be clearly understood that like reference numerals are intended to identify the same structural elements, portions or surfaces consistently throughout the several drawing figures, as such elements, portions or surfaces may be further described or explained by the entire written specification, of which this detailed description is an integral part. Unless otherwise indicated, the drawings are intended to be read (e.g., cross hatching, arrangement of parts, proportion, debris, etc.) together with the specification, and are to be considered a portion of the entire written description of this invention. As used in the following description, the terms “horizontal”, “vertical”, “left”, “right”, “up” and “down”, as well as adjectival and adverbial derivatives thereof, (e.g., “horizontally”, “rightwardly”, “upwardly”, etc.), simply refer to the orientation of the illustrated structure as the particular drawing figure faces the reader. Similarly, the terms “inwardly” and “outwardly” generally refer to the orientation of a surface relative to its axis of elongation, or of rotation, as appropriate.

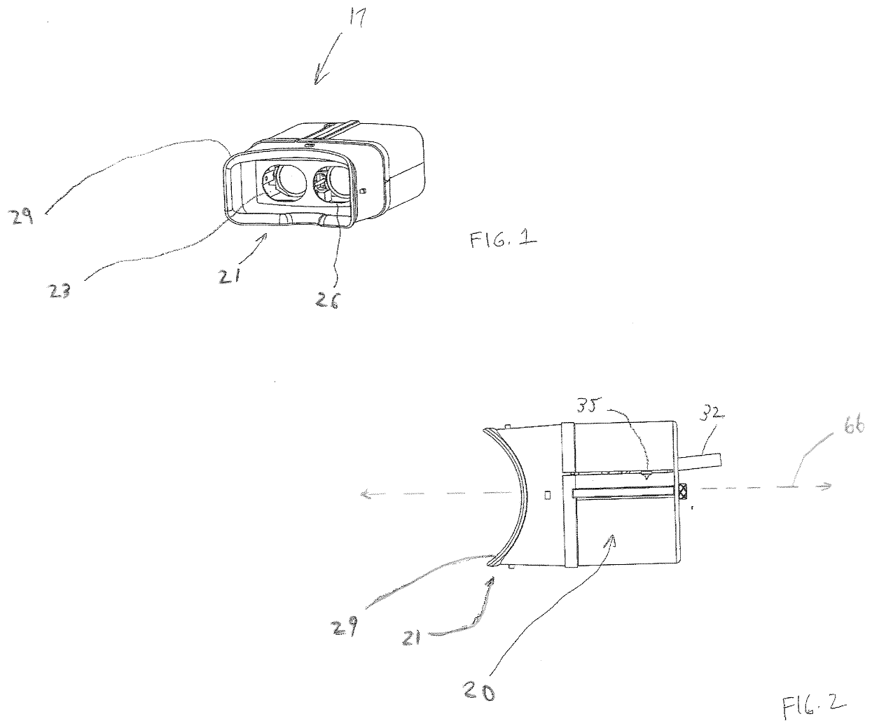

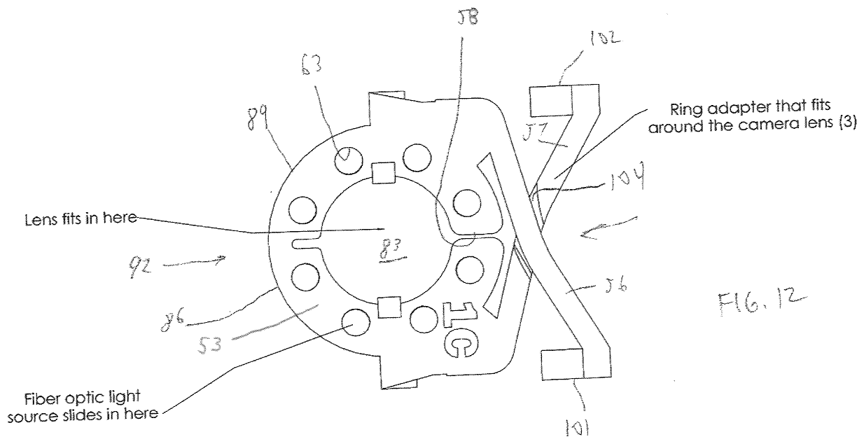

Referring now to FIGS. 1-14 of the drawings, and initially to FIG. 1 thereof, this invention provides a headset 17 forming a chamber 20 that may take the shape of a VR headset or other goggle type structure for covering one or both eyes. The structure isolates one or both eyes to block light from entering either eye. The structure may have at least two inches or more to provide space for a camera. The chamber 20 may only need to provide space to accommodate a camera, an adapter ring, a computer board, and possibly a battery. The front 21 of the headset 17 may be provided with a pair of lenses 23 and 26 covering openings that lead to the chamber 20. The front 21 may also be provided with an elastomeric covering 29 for the comfort of the user. The covering 29 may provide a seal against the face of the user to keep light from the area surrounding the user from entering the inside of the chamber 20.

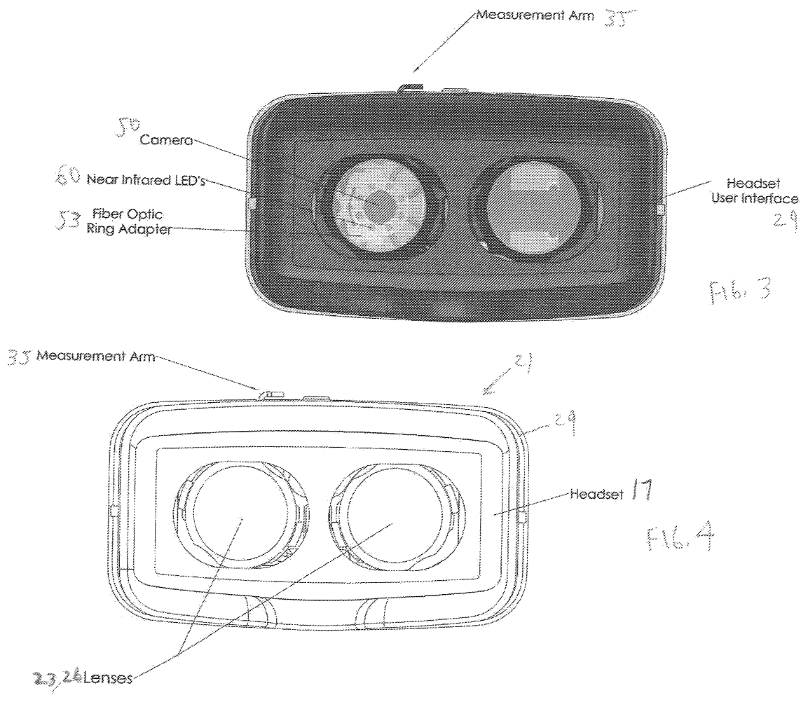

Turning to FIG. 2, the front 21 and the covering 29 may be curved to conform to the curvature of the face of the user. As shown a ribbon cable 32 (described in greater detail herein) may extend from the back of the headset 17. A measurement arm 35 may be provided on the top of the headset 17 for adjusting position of the camera along a linear rail 38 (FIG. 5) disposed inside the chamber 20.

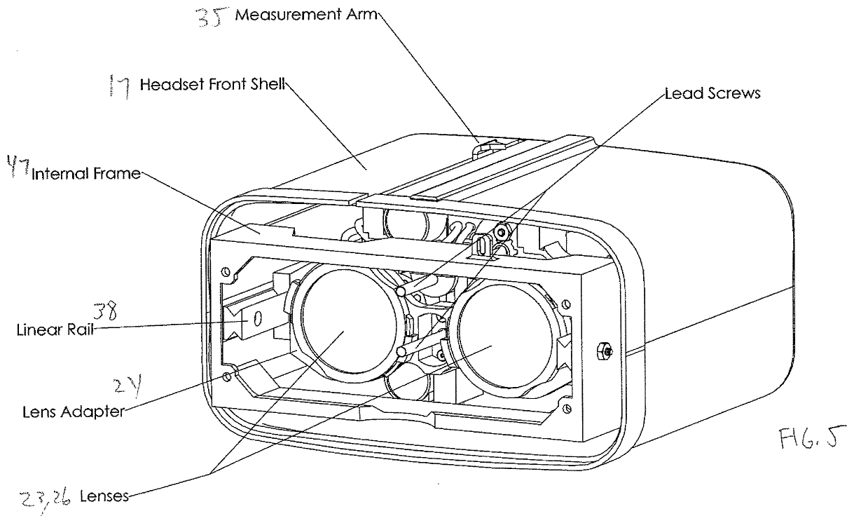

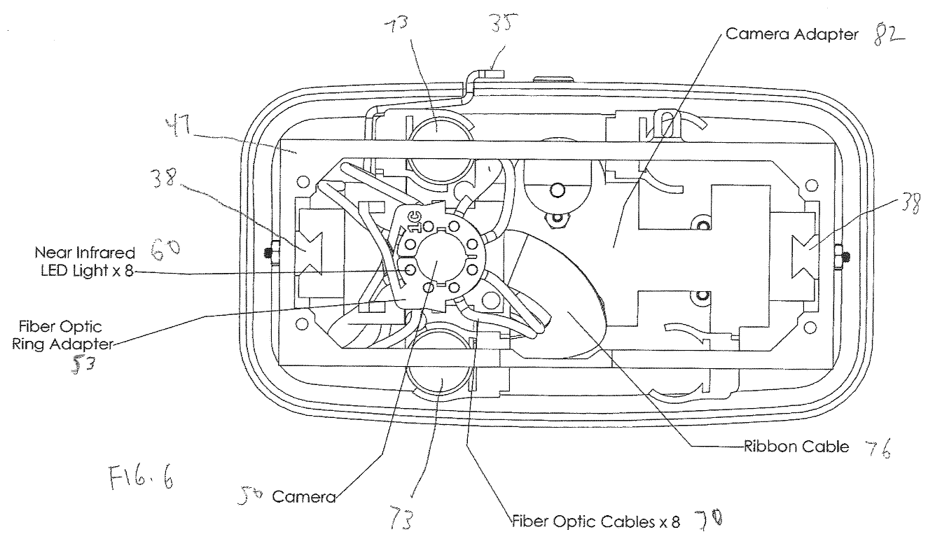

In FIGS. 3 and 4, one embodiment of the invention is shown with a camera 41 surrounded by an adapter ring 53. The adapter ring 53 may have a plurality of LED's 60 disposed therein. The LED's 60 may provide near infrared light.

Turning to FIG. 5, the front cover of the headset 17 is removed for clarity and the chamber 20 is shown in greater detail. An internal frame 47 supports the lenses 23, 26. A lens adapter 24 may hold the lenses 23, 26 in position. As will be described in greater detail herein, a camera and light source is supported in spaced apart relation to the lenses 23, 26 and the position of the camera may be adjusted by means of the linear rail 38.

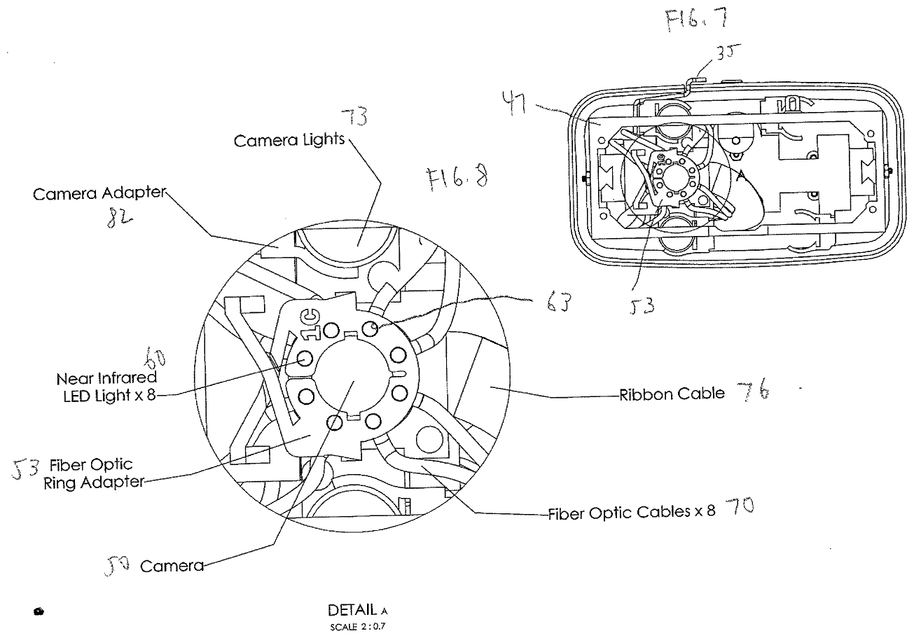

In FIG. 6, the lenses 23, 26 are removed for clarity. A camera 50 is disposed behind the frame 47.

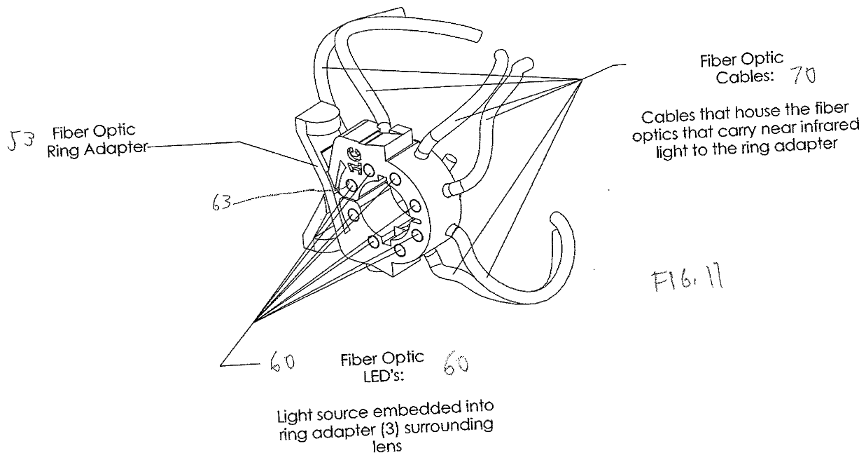

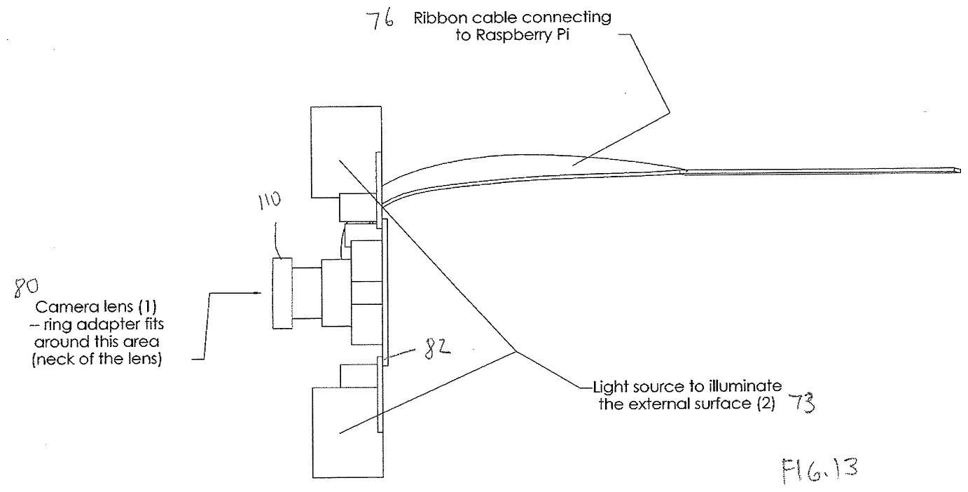

An adapter ring 53 is configured to surround the camera 50 (best shown in FIG. 9). Adapter ring 53 may be disposed around the lens 80 (FIG. 10) on the camera 50. Openings 63 provide channels through which infrared fiber optic cables are connected to light emitting diodes to shine into the retina. There may be various adapters which have the openings 63 at diverging and converging angles, which may be adjustable, to provide diffuse and focused light into the retina. The adapter ring 53 has a pair of arms 56, 57 (FIG. 12). The adapter ring 53 may be split at a midportion 58 (FIG. 12). Accordingly, the adapter ring 53 can be adjusted to fit over the outside of the camera 50. The adjustment of the adapter ring 53 may be accomplished by pushing the opposed arms 56, 57 toward each other to spread open the adapter ring 53.

The adapter ring 53 may be provided with a plurality of light emitting diodes 60 (LED's) that may emit a near infrared light. The LED's 60 may be disposed in openings 63 defined in the adapter ring 53. The openings 63 may be disposed at differing angles relative to the longitudinal axis 66 of the device (defined from front to back in FIG. 2). The elongate openings 63 may be angled to converge or diverge relative to the eye of the user, and the openings 63 provide a channel for directing the near infrared light in a specific direction relative to the eye of the user.

A plurality of fiber optic cables 70 may be configured to transmit the near infrared light into the openings 63 in the adapter ring 53.

The camera 50 may be provided with additional light sources 73. A ribbon cable 76 may provide for outputting data from the camera 50 to an onboard computer such as a raspberry PI disposed inside the chamber 20.

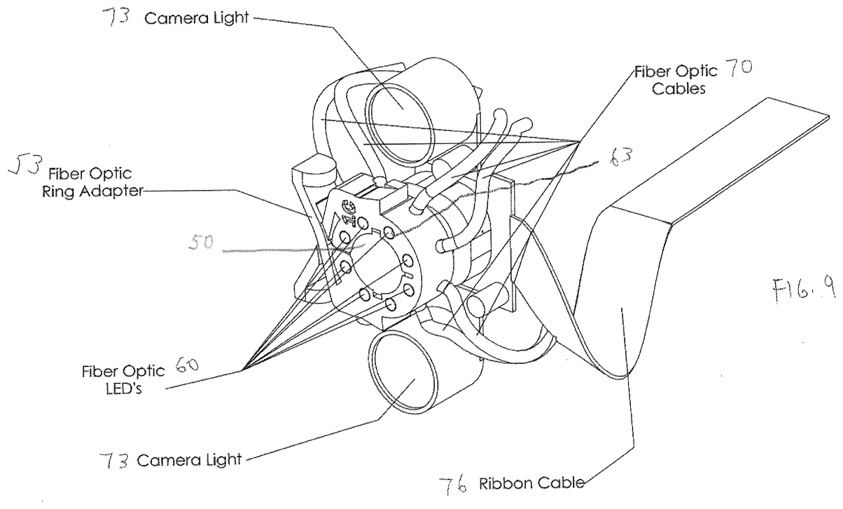

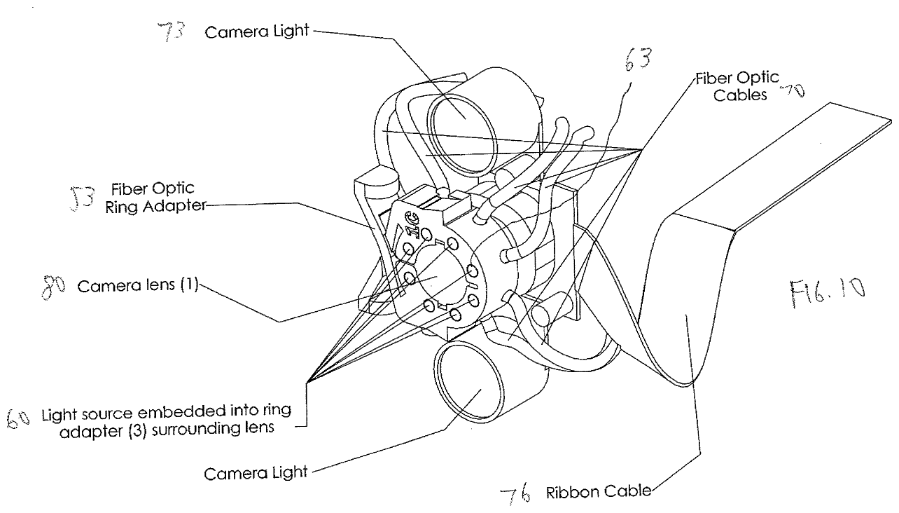

Turning to FIGS. 7-8, a camera adapter 82 may be provided to support the camera 50 inside the chamber 20.

In FIG. 9, a subassembly including the camera 50 and adapter ring 53 is shown in greater detail with the surrounding parts removed for clarity. The fiber optic cables 70 provide a source for the near infrared light emitted from the openings 63 defined in the adapter ring 53. As described above the openings 63 may be angled in converging or diverging directions relative to a longitudinal axis 66 (FIG. 2) of the device. The cable 76 is connected between the camera 50 and an onboard computer. The camera 50 may be provided with additional light sources 73 to illuminate the external surface of the eye of the user.

As shown in FIG. 10, the adapter ring 53 surrounds a lens 80 on the camera 50.

Turning to FIG. 12, the adapter ring 53 is shown in greater detail. The adapter ring 53 is split along midportion 58. The adapter ring 53 forms round opening 83 at the center. The outer surface 86 of the adapter ring 53 may be curved to form a semicircle 89 on one side 92. The other side 95 is also rounded but the arms 56 and 57 may extend in opposite directions from the outer surface 98. Arm 56 extends approximately tangentially for a distance away from the surface 98 and may be provided with a grip 101 extending at the distal end 99. Arm 57 may be provided with a slot 104 for receiving a portion of arm 56 to allow it to pass through arm 57. In order to adjust the position of the adapter ring 53 or to fit it over the outside surface of a round camera lens, the user may press on the opposite ends 101, 102 of the arms 56, 57. As a result, the central opening 83 becomes larger as the adapter ring 53 expands where it is split apart.

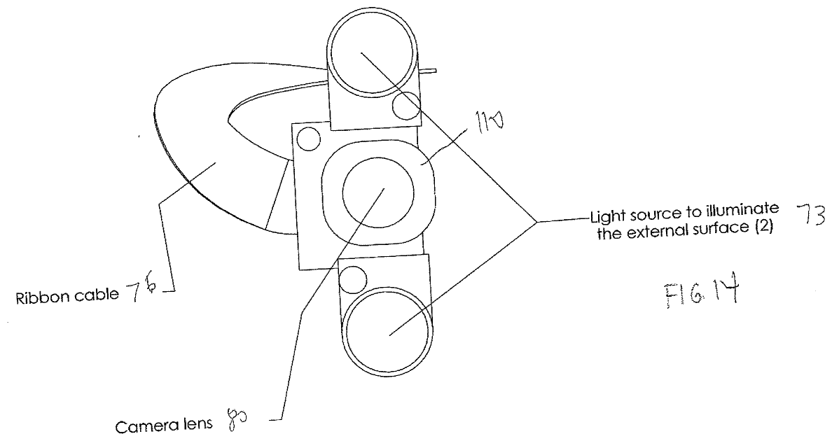

In FIG. 13, one embodiment of the camera 50 is shown. Lens 80 is mounted in an enclosure 110 that provides the mounting surface for the adapter ring 53. Camera adapter 82 supports the elements of the camera 50 for configuration inside the chamber 20.

The device of the present invention may be used according to the following steps.

### Process

1. Subject wears a device creating a darkened chamber 20 such as a virtual reality goggle to obtain dilation of the pupil.
2. Infrared light source 60 is projected into the retina to achieve illumination.
3. Infrared camera 50 captures image of the retina.
4. Captured image is uploaded via onboard processor such as a raspberry pi using existing internet connection to a server.
5. Server maintains an algorithm to render a diagnosis.

The present invention contemplates that many changes and modifications may be made. Therefore, while the presently-preferred form of the eye imaging device has been shown and described, and several modifications and alternatives discussed, persons skilled in this art will readily appreciate that various additional changes and modifications may be made without departing from the spirit of the invention.

What is claimed is:

1. An eye imaging device, comprising:
an enclosure defining a chamber, the enclosure having one or more openings in a first surface;
an adapter ring disposed inside the chamber in spaced apart relation to the one or more openings in the first surface, the adapter ring having a frame with a central opening defined therein, the central opening configured to receive a camera, the adapter ring having a plurality of openings defined in the frame surrounding the central opening;
one or more light sources configure to emit light through the plurality of openings in the frame;
wherein the plurality of openings are angled in converging or diverging directions relative to a longitudinal axis of the device.

2. The eye imaging device of claim 1, wherein the one or more light sources comprise light emitting diodes.

3. The eye imaging device of claim 1, wherein the one or more light sources emit infrared light.

4. The eye imaging device of claim 1, wherein the one or more light sources emit near infrared light.

5. The eye imaging device of claim 1, further comprising a supplementary light source disposed inside the chamber.

6. The eye imaging device of claim 1, wherein the adapter ring is split.

7. The eye imaging device of claim 6, further comprising a pair of arms extending from opposite ends of the adapter ring.

8. The eye imaging device of claim 7, wherein the pair of arms are configured such that pushing the arms toward each other causes the adapter ring to spread open to accommodate different size cameras.

9. The eye imaging device of claim 1, wherein the one or more light sources comprises a fiber optic cable.

10. The eye imaging device of claim 1, wherein the camera is an infrared camera.

11. The eye imaging device of claim 1, wherein images from the camera are transmitted to an onboard computer disposed in the chamber.

12. The eye imaging device of claim 1, where the camera is configured for transmitting images to an external computer.

13. The eye imaging device of claim 1, wherein images from the camera are uploaded to an external server via a connection to the Internet.

14. The eye imaging device of claim 1, further comprising a rail disposed in or on one of the adapter ring and the enclosure and a groove configured to receive the rail in sliding engagement, the groove disposed in or on the other of the adapter ring and the enclosure.

15. The eye imaging device of claim 14, further comprising a measurement arm disposed on top of the enclosure for adjusting the position of the camera along the rail.

16. An eye imaging device, comprising:
an enclosure defining a chamber, the enclosure having one or more openings in a first surface, each opening covered by a lens;
an adapter ring disposed inside the chamber in spaced apart relation to the one or more openings in the first surface, the adapter ring having a frame with a central opening defined therein, the central opening configured to receive a camera, the adapter ring having a plurality of openings defined in the frame surrounding the central opening, wherein the adapter ring is split;
one or more light sources configure to emit light through the plurality of openings in the frame;
a pair of arms extending from opposite ends of the adapter ring bordering the split, the pair of arms configured such that pushing the arms toward or away from each other causes the adapter ring to spread open.

17. An eye imaging device, comprising:
an enclosure defining a chamber, the enclosure having one or more openings in a first surface;
an adapter ring disposed inside the chamber in spaced apart relation to the one or more openings in the first surface, the adapter ring having a frame with a central opening defined therein, the central opening configured to receive a camera, the adapter ring having a plurality of openings defined in the frame surrounding the central opening;
one or more light sources configured to emit light through the plurality of openings in the frame.

18. The eye imaging device of claim 17, wherein images from the camera are transmitted to an onboard computer disposed in the chamber.

19. The eye imaging device of claim 17, where the camera is configured for transmitting images to an external computer.

20. The eye imaging device of claim 17, wherein images from the camera are uploaded to an external server via a connection to the Internet.
